SEQUENCE LISTING (10 sequences)

SEQ ID NO: 1 - protein, 295 aa, Gallus gallus
MHVKAKNLLRMCKWLKGLRNARGSTWGRSGGRKPMRSSGRLPRSADPGWGNHANRAAVVTRGISSHSPRVCLCLCQDAPTSEELEQFAKDLKHKRIMLGFTQADVGLALGTLYGKMFSQTTICRFEALQLSFKNMCKLKPLLQRWLNEAENTDNMQEMCNAEQVLAQARKRKRRTSIETNVKGTLESFFRKCVKPSPQEISQIAEDLNLDKDVVRVWFCNRRQKGKRLLLPFGNESEGVMYDMNQSLVPPGLPIPVTSQGYSLAPSPPVYMPPFHKAEMFPPPLQPGISMNNSSH

SEQ ID NO: 2 - DNA, 888 bp, Gallus gallus
atgcatgtaaaagccaaaaacctgctgcgaatgtgtaaatggcttaaaggattgcgaaatgcccgcggcagcacttggggccgtagcggtggcaggaagccgatgcgctcgagtgggcggctgccccgaagtgcggatcctggctgggggaaccacgcgaaccgcgcggctgttgtcacccgcggtatctcgagccattcaccgcgtgtctgcctctgcctctgccaggatgcgccaacctcagaggagctggagcagtttgccaaggacctcaagcacaagcgcatcatgctgggcttcactcaggctgacgtggggctggctctgggcacgctctatgggaagatgttcagccagaccaccatctgccgcttcgaagctctccagctcagctttaagaacatgtgcaagctgaagccactgctgcagcgttggctcaatgaggcagagaacacggacaacatgcaggagatgtgcaatgcagagcaagtgctggcccaagcccggaagagaaaacgcaggaccagcatcgagaccaacgtgaagggaacgctggagagcttcttccgcaaatgtgtgaagcccagtccccaggagatctcccagatcgctgaggacctcaacctggacaaagacgttgtccgggtctggttctgcaaccggcgtcagaaaggcaagcggctgctgctgccctttggcaacgagtcggagggggtgatgtacgacatgaaccagtccctggtgccccctggtctgcccatcccagtgacatcccagggctacagcctggcgccgtccccccccgtctacatgccacctttccacaaggccgagatgttccccccgcctctgcagcccgggatctccatgaacaacagcagccactga

SEQ ID NO: 3 - DNA, 51 bp, Artificial
  Interfering RNA
aagatgttcagccagaccaccttcaagagaggtggtctggctgaacatctt

SEQ ID NO: 4 - DNA, 51 bp, Artificial
  Interfering RNA
aacagaaaccttcaggctgtgttcaagagacacagcctgaaggtttctgtt

SEQ ID NO: 5 - DNA, 51 bp, Artificial
  Interfering RNA
aaggccaagagccgcacagctttcaagagaagctgtgcggctcttggcctt

SEQ ID NO: 6 - DNA, 51 bp, Artificial
  Interfering RNA
aacgcagttcatagcagttacttcaagagagtaactgctatgaactgcgtt

SEQ ID NO: 7 - DNA, 47 bp, Artificial
  Interfering RNA
gcggcaaagccgacaacaattcaagagattgttgtcggctttgccgc

SEQ ID NO: 8 - DNA, 51 bp, Artificial
  Interfering RNA
aattggctcatcctggatagattcaagagatctatccaggatgagccaatt

SEQ ID NO: 9 - DNA, 51 bp, Artificial
  Interfering RNA
aagggcttcagagacaactatttcaagagaatagttgtctctgaagccctt

SEQ ID NO: 10 - DNA, 12115 bp, Gallus gallus
ggtggaattcgaaagaggaagagtgagacattcaaagtggtggtgtttgtctccccagcaaactgttacatgagatgaggcctgcttttgtgtaagtggctgagcagctgcccgccaatggaaaatagtgaatgaattctttgttttgctttgattgcatgcacagctttcacctagcaaactgtatctcttcctacaagatctagcactttcacctttccgattctcttccctctgggaagagcaaacaaacaactgtgtggtgctgagctgcctgttggattatactacagcagtaactgagtttgagtgcaaatttcagtccacaggaaactaagatgtcccatgcccaatacagggaacagaaaagccacagtacataaagtaatagcagtgaccataaagagcctacatgtaactggcttcaggtcattttcatcttctctgttcataagaacaaggcacccaagcaaagaacacaacccccaagtttctagatcttttccacagcctcatatgcatatgtcacatggtcaaatagcaattgtctagttacctatggaagcaacaagagtattctcatgtttcatgaattgatttttgtctctgacagtccaagagagattgattctgattcctaaactgtaatttctttgttactcttgagaaatattactcaggtgtaccgtttatacttggactatcagatctcatttaatatgtttatttttgctattacattagatacagcattacattttctattttatttccttgttcacccctcacacactgggcacagaagaatctgtcaggaactgaactgaatatgatgtcacttcttgcttactgttcgaccacagattgatttacacagcaaatacaactcaaaaagaatggcttgaaatagaaaattcatcatagtattatagataatcagaatatgaacaactgattcagaatccagagccactgttttctgtgaccaagtcacagtaggttttgccagatagtactaggttatagcgagggtcagctactccctaggggatgagaagtcatgtgcccaaagtagaactgttgcatgttgaagccaaattaccacctaagtgcctgacatggaaaccggtgggaaagagatccatgtttgagaccagcctcagctttgctgactcacatgagtggtgacttgagttggtctctgtgtgtgcatgtatgcatgtgtgtaaagcttttcctgatatccctcacttgattcacttcataaattgcgcttagaggtgtcttatttctgctgttcttcttccccccataacaacgaatgtgatccattctctctgcttctttaatacaatcttgaaagcaccagttcttgttttcctaaggttgaaaaatctgtatgtgatcaagggggtgcatccaacaaactggattttacttaacagaatagtgctactgttctgtgagttacaggtagtcatctttcagtttctgtaaagtcaaaatgtctttaggttatatctgtccataactctgagtaattcttttgactgtgttaaaagattagtcatataaatgagagaagaatcagatccaattgtagctttagagtttaatgactgaaatgggcaataaaaaatgaatatttattttagtcttgcaaaaataaccttgccttgggaaaagcaaacacagaaagattatattcccaagcacatagtacagagagcagggctgtacagatgacagtacagaggaagaagagtagtgggccttggacactgttcttcattatgcctgctacctgcctccatttccacatcctgaggtttctcatcaaggatatttagcatggtccagtgtactcaagaccctcagtgtcaagctgactgtgcacaatattggtaaaaagtgaggaaagttggcaacattttatgacttctgtcttctaaacatagtaaagtagatgtccattctctgtggaatagataatcctgaaacaaaaactaaaatatgacatttagatccttcaacagttacactttgaatttgctgtgatttaaaatggacatagcatttgtaatagcacagaatatttcctttccactggtgtcacaaaaagtggatttcccagatgtgcaatgacctacttaactataaatttatttcattccatttcactaaaaacaacatgtcagagaactacttagtcagattttttgtttggttgtttggtggtttaaaaaaaaaaacaccaaaaacccaagggtgtgtgcttctttgcaagtccaaagatctggacttattcactgaagtagagcctctcatatccaggggcacaatcatttagcaccagcatcttcaggaacacaatcactctgctctttcgtgccatttaggaagcagcctaagcatctgatctgtgcaatgggcctacatccttgcaatgcacagcatgagccatgctcagtaccacctgcaatgccagtgacgcatatgctgacaagaaatcacagctgggggcagtctgcagtgtcctgtctggatgttgccatgggcccacacatgagctgggacatatctgtgtttgtcctgagttttaagacacagtaaaatacaacataagctaggaaagctttctcttgacttcaaaaagcatcaccatgatgatttggtttttaatctgactcacaactgaaacagggtccctccatccctttcctgccaaaatttcactgattttttcacctacatactgtattatcctccaaaacaactaatagtttgtagctttcagctggaatgtcataggaccaggtttaaaatcatagaatccttagagttcaaagggacctctgaaagccatctagtctaactctcctacaatgaacacagacaacacagccagatcaggctgatcagggcattatccaccctcaccttgagagtctccagggatggggcatcaaccacatctctaggcaacctgttccagcgcctcaccaccctcactgtaaaagattttttccttatatctaacctaaatctaacctccctgagcttgaaaccatttttccttgtcctgtcacaacagaccctgctaaagagtctgtccccttctttcctgtagctccccttcagatactgaaaggccactatcaggtcaccttggagctttctcttctccaggttgaagacacccagttctctcagcctgtcctcacagaagaggtgttccattccatggatcatttttgtggcccttctctgaacatgctccaacaggtctgtgtctctcctgtactgaggactccacatctggatgcagtagtccaggtgaggcctcaccagtacagagcagaggggtgggatcatcaagcttccctggcttctttggcttcttgccagggttgattctgagtaaggggaagaaaatgttaggtaaatcatgttatgtggctctcttgtccaacatgcagacaagacagcagaggagcatgacaagtagtagatgggaaggaaacccttctcccatctgagctgaaggctttaattttgttcgggttcacaatactttccggatgtccactggatgtatctcttctctcaggaatagccttcagcagccaatcctattgggaacacacaggaaaaaaagagagaagaacaaaggaaggaagatcataagactcaagattttattcatgtgagaacagttgaaaatgctatgagatgtgctccttcttcctagaggagacttcatttgtcttcttcatttttatgacatttaatttgtcctgtccttcacttgtagttcaatttgagtttatacatccttgaaagagtcacttgtatgtggaaagtgagggccaactttgtgaagatctctccagtttaagcctgtcccaccgtatcagtatggagcagatgttacagagatttgtgtctttggtgccttgctgtggtgtacaagcaatctggtgatcaagaggtcactggagcaggaagtgagtatagagaatatcctgagaggagaagtggtgagacctcacataccttagccactgttgtcatttagcaaatacagctcttctttgtgaagtcaccagcttgagatctcctggagactaccattcagaaatgggaagaaaggacacagcaacattgagtgtttctcagccccttgtttcaagagacactggtgcattaggagtgaacctctccaatcctgggttttttgtgaaggaaatgactagcatttttttcactatgtgccagtcaagtgggtcatgctccatacacagccttgaacgtggctccccatagctctagggttatactgaggtctctgtacccaggcttgtgcttcatctcaagaggtgcaggaagaggtgcaggacatcctacctccacctcatctagctatctctgctttttccagtcccttgattttcctctccccatgacttcacctctctcccagggccacagaccagtgaagctgaagcacaactctgttgcctctccagtagaggccagggggagtgggcaggcaggcaatacagcccccaagacacgactgccacaagctcagtggtggtcatagcagggcagtggtgaggcagccctattgcttgacattactttgattatatcattcagcactgcgaggaacatgtttaagtgctcctcatggctgcctcaagtcatttccatgaaagacatgttcaggaagcaggcattgaagccagtagctggtgtacaacctattctctggagatcaaaacatagaagaggcatgatccagctgcttatgcaacaggagcaggtcttaatcactgagttgtgctgggaaacacacagcctaatgtcctgctcttgctgcagggcgccaatactgtctagtccccatagtgggtgcatctagtgggcagatcagaaatcaccaccagctttggccagcacatgggattgcagagcaggtgggtactgaaagaaactgaaatctccagtggtttattttaactcactgtctgtggactgcagtggctgagaagagattaactatatcctgctggaccatttctacagcagcagtgggaacatctggcagctgcagtaaatgaagaaagataactgaggtgttaagcttgtcagagggccctgctggatcccaggccaagacctgtatattcacaccaaaagtatgcaacactctgcaaggttactttgcatctgcagatgctttgacactattaatagattgtttacaactatagtggcatgatttagtcatgagtgggattcagataaggtgttctcaggcctgtcttagccccagagtttgcatgcaaacatttcacagctgacttcagaactggaatttttagcaagatgttgttcccaatagtcgtggctgctcctcctcctccttcatgactaagttgtactgaggaagcatttttatattctcacagatgagtttgagtttctggagttctgctattgtatgattctgtgttttttcctgatcttgcagcctgggaagacacccagtcaaatacgcagtctctatttctttgctgctcacaaagatgtgttttcacttcctgatgtaaaaagttcactctggacaattcgaaggagatgaacagatcatttttacctttgttgatttgaagaagcaggccacaatatattttgtaatgtatttttagaaataattgatttggaaaagtagatatgccatatatttcagtagtgtatttttagaaatatagttgttgaactgtgttatgtgattagttcttgcttgcaaaactgtaatagcttttagatgttgtgaatagtaagaatgtagcattattaactagaaagcatgttgtaaagctattctgttttgacaagagaccctcagcaaaggagacacaataatgaaagccagggcctccacacaaggctgaattgtctcactctgcaattaggttgggatgcaacacgcagacattaagatactcccctctatcctccttatcttcaacaacctagtatatatcattaaaagacagagtgccaaacttatctcgatccaaggatgaacaggtgtccagaaataatgtctgagtgctgagagagcgaatgttacgattttggtacctataaattacttataataggcattctcctactgagcatgcatcaatggagaaagttcatctagatgacaagcaaggaagaccattggatgaagtaaaagactgtctcttggactgtcatgatgctaaaaggatgctagacagtagaaaagaccatggagtaaaaaactggaaacctcataaatcactacctgagataggcgtgtataattgtctcacgcataattgatgagtatgtactatccgtggaaatataatggattctgaatcatgtaatctttgaagcacttatggtggaaatatccccagagtttcccagtgtcactaataaagaatacctgacttaacagtaacaaaactttgctgttaagtttctacacatcgatttcttcaattcaatgtaaatgttatatcctcttcccaagcctccaacttccactcaattcagttcatagaatggcttagaatggaagggatcttaaagctcatccatttctaacaccctgctgtggacaggattgccatccattagctcaggctgcacatcgtcccaattcaactcggccctaaacacctccaggaacgggccatccacaacttttcaggacaacctgttccagcgcctgaccatattctgagtaaagaatttcctcctaaaagctaatagaaatctttcctcttttaatttaaaaccatttcctcttgctctccctctatcagactgtgtaaaaagtctgtctcgatcttgtttataagcttcctttaagtactaaaaagcaattcctaaataaaagacattctgttaaaaaaaaactactgtcgctcactgttcttccttttgtctctttatctttcccatctcagcaaagtcggccacataaatattgttagacaaaagcaggtaaatccttttcttccgtcagtcccagacaatatccacttgcagatcccatagaacagcactcggtttcaccggctgccattcagatgtagagcgggtgccagccagggagcctcaggctctaacagttgctaatcacacatgaatgtgtgtgcaagagccagtcttggaggaaaggagacggtggaccaatggcggtaaggaccagacagtgtggaggtctctgtccctacctgtgagaaatttgaaagagaagaaccctttctcagtcatccgtgtaaccagacacttgccttataggtgagctgctcctgtggggactttctgggcttcagaaatccagatgtaacagaatgggcacgatgaattgctcgagaagtatgtgctaattggttcatgagatatattaggaataaaatgactcttagatggtgtgcctacttatggcctgcacaagcataattacagtcatccagttggcacgtaatccccattaacaacactgaaaaacgtgggattagcttcggatgaaagatgtggaagaaaacaggtgccacaatacacagcgaacccaagagaacattcaacgcacatcctaccactcaaatgccccactaactgtaggctctgagtagctatggactttgcagacacttttcccactgaatgggttctgtaaaattgtccccaccagaaagagtaaagaaacctgaagaaaaaaaaaagcctctgtttcatttaaaagtttacaaaaggaatctgttcagggaagaaatcacaaactgtctgatacagagcatcctttaggaggagagcatgggaggtgtcacgtatttttaaattacacggggtctttgtacgacgagaaatacagcactaggtatccccccctggtggtggaaatggcagaaactttcaacaaagcaagcacagccatcactgtgcatttgtttttagtaagaggacatctaactgagcctatgagaacaatcactgttgtcaaaagggagaggggagaagttgatcagggagcccatttggaaggttcttgtccaatacgcagtctctacatgttccccagtactgaacaaccctggctctcaagtactcctcagggagatcattctttattcgagttcataagcactgataggaaatgctttctgagacatgtacttgcaaatacagcacttcagcatttctgtgtgtagcaagcattagtgtaaagaagggctgataaagaacaggcccatctgtgttgtctcactagctttctttccaccttattttgacactccagattttctgccggcctgcctccaggttttatgccacggttcctctgttcaagtctgcctctccacggctaacaatgagtcaaccatcaaccagtaattctcaggtgattaagcagcagggttgtcagtggaaaggagaagctttgtccttttcttgacctgttcccctaaaaagtcaagagaaagaaaagctgtatttcaatatgaatgaaggtctaatttttattattctttgttggctgccttccttccacaaacatgacatttttgatccagactgctagctgtgaccaagccatgccggggtggctgtctggagtcaagggctgtggaacagatgttttctgaaggttaactgaagagagttcaacctaaaaaataccaagggagactgcttgcaggaaaaggtggacaaactcaagagaatacaatgaacccatttcttcctgcatttcttcttcccccatcatgcacatactttacagagcatgatggctcccacagaaacctctgaggagtaacaaggctcagccatgggcccctcgttacagctcactgagcaccatgaacatgaacaatacaggaactctcagtgacacttcaaaatattcactaaaacacagatgagtcacgtttgagtcagttgcagaagaacacttaatctacataaaatttgagacacctctaccagcataacagtgacccactgatgaaaatgaacttagcagtgctacgaggtaggaacccatagaactctgatccagcagtacaagctccgaagctgaatttacagtcagctgctgcttccccagcccctgtaaactggtctgtctgtatcactttagtgtaagtttgagctccatccctctgaaagcggtgaccctacttacagtagtaagtgaaccctcgtagaaggaacttgcatgaaaacaccttccagcccttgtccaatctcacctgtgtttcagccaagagagcttttacacatcagcttctaccctggaaggactatggaaaaggctccttgggacatacttctgtcaagtgactgcttacattcccttccataggcttaatgacccagctgatgcttacacagtgactaatgctccataatattaggcacataatgaagtgataactgcaaaagcagggagtggccacagcattaagagtgaggtgacacagccttcagcttaaaaacaaatcaatttcaatctgttgggaagaataggaccaaaacagatctgaattgcgttgctgctccacctgaaaacagcagcaaaaaggaatatgagcgaatatttggatctgctctcagagtcattctcagcaggtgtttttaaaaaatcatcatctctaaatattaatgggcagtttatccactttgcagcttaatggtgtcacagggcatatgtcccagatccagcacctcaggaagcctcttgaatgtctacatcctgcttcctatagcattctctgggcctgtatgacttatggatttaccttctcaatatgttattatgtgtaaaatttattttagggaaaataactgaatttcttttcataaattctgactttagcgctagacctgattttatacactcacacaagccttatttctcctataaagacatttcaaaagataagtgataatttggtaagagtcctgactgaaagcaaactgatgtcactagagggcgacacgtggaacaaaaaaacagtaaatataaagcaaattagagacaagtgagatgagaaaatcaaaagataaacaaagattgtgggaaaattaaggggaaaaaatgaaatcaattccatgcagacagcaaagcaggtccatctctgggagggaaacagctcaacagcaaaaaaattataaaccaagggagactggggatttgatggcagatgaggtgttagaggctgtaaaatgctaatccttctgctatgggtgcccctagacaagctgtgtcacctctgatgactcagtctgcagcttgtgggatggcaagagtgcatcgtggaacttctcagcatcgtgtggcatggcctccagtcttctgtctgtgtcaatagtatttactatttcacttaatgcgcactgtagaggcaaacaaatggagccccttaagttattattgccaagagacaaacaagtgctttggtttgcaaacacaacttccctgacattgaaatcctgtgtttttccctctctttgcaataggctgtttttctatgagctcagacattgtttcacaggtagcacacatatctccagggaagccctgtcagcaaagggactgtgcagatcagtggccaccacaaccatatcggttacatggcaattttgcacatttgacagataaaccctttctctgtgtgcattggctaagcccataacagcaagcaagcaaccgtgtgtgaaaccaacagccgaacatgtggggacatggaagagcctacgggaaacggaacgcacagcttacaccacgtgcttcagggcaagacagcatctgcctccacattgtacccctcatcgaattataataacttgctcctgcatccctaatcacctctctacctggaccctacccagattctgagaaactaaaacaccataaattataaagtcataaaaaatgagctcaatgagggcccctccaagttgcaaaagcaccttcaggtcagcaactccctgccactacctagcaactctggagaaagggggcttaacgtaacacatgggggtgtctgctcatcatgactaagctggggagggagaaatatatggaagtaaggggggcctatttctgttgcctctagaagattattagcagctaatatctgttcctcatttcttttcagacagggaggagcctgtgtcctgtcctttcactaggtgaagtagggaatttgcttgatttgtcatatgctctcatttgtggatctctcaggttgttttctgcattttataaccccttttttgtaaactcatgcttatttcaaatgtcatgaaaagaattgacaaatatctgaggaaagatgctgagaagtgacacttctttgtgctcctccaaaatagatgtctgtctcatccttgggttagcatttagaaaacctggggagctcgtctttgagtttgcttccctgtgatgcagacaccatcctagaataccgccctccccgccggccccagtctgtattcccttgcaaggaggaatgcttatctgtttacaatgatggctgcagtctgcaatgcagacacgaaaagcctccgcatgtcccgggggggtctctgggctccccctccattctttgtacttgggtggggaccgatgaggaggcgtaggtgggtagcaagactgttaaatgtgagggcgggggtgccagcccagcccgccgcacgggacggccttgctcacacctgctaggcctgaggtggaaacaaggccccctcctcccagccccggggggtcgggtgtgcgggcagcaaagaactgactcccaccgaggaaccaaagggggaagctggccttgctgtcctagtcctcccccactctccgaatatccccatagccactcccctccaatcaactgacttttaacatgataatggtcgtgacaatctcttggctaaagccagtctggggaggacataaaacattgcccctcaccgcctaatggggtatctccgtgcagtaggcgcgtatgcaaccagctcaccgccagcaacggctggaggtgctgtcccgaccatg

(19) United States
(12) Patent Application Publication
(10) Pub. No.: US 2009/0311789 A1
(43) Pub. Date: Dec. 17, 2009

(54) METHOD FOR PREPARING DIFFERENTIATED AVIAN CELLS AND GENES INVOLVED IN MAINTAINING PLURIPOTENCY

(76) Inventors: Bertrand Pain, Clermont-Ferrand (FR); Fabrice Lavial, Saint Etienne (FR); Jacques Samarut, Lyon (FR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/294,030

(22) PCT Filed: Mar. 19, 2007
(86) PCT No.: PCT/EP2007/052572

§ 371 Date: Sep. 22, 2008

(30) Foreign Application Priority Data
Mar. 24, 2006 (FR) 06/02597

(51) Int. Cl. C12N 15/87; C12N 5/06; C12N 1/21; C12N 15/00; C07K 14/465

(52) U.S. Cl. 435/455; 435/349; 435/252.3; 435/320.1; 530/350

(57) ABSTRACT

The present invention relates to a method for preparing differentiated avian cells from stem cells in culture. Genes involved in maintaining the pluripotency of avian stem cells were identified and cloned. By inhibiting the expression of these genes in stem cells, the latter lose their pluripotency characteristics and enter into differentiation. These differentiated cells obtained in vitro can serve as host cells for pathogens, in particular viruses, and can thus be used for the production of antiviral vaccines.

This invention relates to a process for preparing differentiated avian cells from stem cells in culture. Genes involved in maintaining the pluripotency of the avian strain cells have been identified and cloned. By inhibiting the expression of these genes in the strain cells, they lose their pluripotency properties and become engaged in a differentiation pathway. These differentiated cells obtained in vitro can serve as host cells for pathogens, in particular viruses, and thus be used to produce antiviral vaccines.

A stem cell is a pluripotent or multipotent cell of embryonic or adult origin that has a capacity for self-renewal. In other words, a stem cell is a non-cancerous cell capable of dividing itself indefinitely in culture and producing a daughter cell having the same proliferation and differentiation capacities as the mother cell from which it originates, and which is capable of producing differentiated cells.

Chicken embryonic stem cells (CESC) have been isolated by culturing stage X blastoderm cells of chicken (Pain et al., 1996; patent application N^{o} FR 94/12598). These CESC have all characteristics of embryonic stem cells (ESC). A culture medium allowing the maintenance of the pluripotent character of these bird cells was the subject of the patent application WO 96/12793. Most of the characteristics of these cells are described in the publication of Lavial et al. (filed).

One of the properties of stem cells is their capacity to produce differentiated cells both in vitro and in vivo. These differentiated cells from a homogenous population of precursors are obtained by modifying the culture conditions. Indeed, the CESC cells remain in proliferation in an undifferentiated state only under specific in vitro culture conditions, in the presence of growth factors and cytokines as well as inactivated “feeder”. Once the medium has been depleted, the proliferation balance is modified and the cell engages in differentiation. One of the advantages of obtaining differentiated cells in vitro from CESC cells is the very wide range of phenotypes that can be generated from these cells. Indeed, the CESC cells have pluripotency properties; they can be differentiated into all lineages defined at the embryologic level, into mesodermic, ectodermic or endodermic derivatives. This pluripotency property, specific to a stem cell, is not present in the primary cells of a tissue that are already engaged in a differentiation pathway. This property is maximal in embryonic stem cells and is present but more limited in adult tissue stem cells, which are multipotent. The latter can be differentiated only in differentiation pathways belonging to a single lineage.

Nuclear reprogramming processes enable a differentiated cell to recover its differentiation plasticity. These mechanisms are beginning to be decoded at the molecular level in particular by understanding the epigenetic modifications that occur at the DNA level (methylation on the CpG islets) and/or its components such as histones by acetylation and deacetylation, methylation and demethylation, lysine ubiquitination, serine phosphorylation and proline isomerization processes, of which one of the consequences is that numerous protein complexes are recruited, which control by their association with promoters, the expression level of key genes. Direct post-translational modifications of these players are also an additional regulation element.

Various processes are known to a person skilled in the art for inducing or controlling the differentiation of pluripotent stem cells into differentiated cells in vitro.

A first approach consist of growing cells in a culture medium containing few or no cytokines and growth factors necessary for maintaining cell proliferation. In particular, the absence or low concentration of one of the cytokines of the family gp130 (LIF, IL-6, CNTF, GPA, IL-11, etc.) induces slowing of proliferation and a progressive loss of pluripotency markers.

The differentiation of cells subjected to this process is not homogeneous; various cell types will be obtained depending on conditions of density, autocrine and paracrine secretion of cells, and their relationships with one another. The heterogeneity of the cells obtained can be estimated by the detection in the whole culture of most of the early lineage markers, such as, for example, Brachyury and Goosecoid genes specific to the mesodermic lineage, certain Pax and Sox genes specific to the neurectodermic lineage, and Hnf3 specific to the endodermic lineage.

A second approach consists of producing embryoid bodies by growing cells in a flask that is not treated for the cell culture. The dissociated cells are suspended either in a large-volume of depleted medium (less serum—from 0.5 to 5%, for example—and an absence of growth factors and specific cytokines) and subjected to slow agitation, or in a small-volume depleted medium, which is then used in the hanging drops technique. Various examples are provided in U.S. Pat. No. 5,456,357, U.S. Pat. No. 5,914,268 and U.S. Pat. No. 6,458,589.

Another process is the formation of embryoid bodies directly in a cone-shaped tube (Kurosawa et al., 2003), on a specific treated surface (Konno et al., 2005) or by encapsulation in alginate microbeads (Magyar et al., 2001). By thus preventing adhesion of cells and their basolateral polarization, the embryonic stem cells proliferate and adopt a three-dimensional structure that imitates the various germ layers (Dang et al., 2002). The cell types thus obtained are very heterogeneous. Moreover, heterogeneity is observed in the kinetics of obtaining the various differentiation stages.

A third approach is the use of chemical differentiation inducers. By “chemical inducer”, we mean any non-peptide chemical molecule not related to a growth factor or a cytokine, whether of natural origin or obtained by chemical synthesis.

For example, DMSO (Dimethylsulfoxide) is used as a general inducer that allows the obtention of mixed differentiated populations with a plurality of derivative (Dinsmore et al., 1996). Retinoic acid allows, alone or in combination with AMPc for example, in a non-exclusive manner, the obtention of the differentiation of stem cells into mesodermic derivatives in particular, with the obtention of adipocytes under specific kinetic conditions (Dani et al., 1997), of cardiomyocytes, and of various muscle cells characterized by a contractile activity and the specific presence of myosin (Rohwedel et al., 1994; Drab et al., 1997).

Most of the protocols currently published and that enable the obtention of cells with a specific phenotype from mouse embryonic stem cells associate chemical inducers and complex combinations of growth factors. Variations in the kinetics of inducing and contact with these agents make it possible to modulate the phenotypes obtained. Examples are provided with the kinetics for obtaining adipocyte cells (Dani et al., 1997), osteoblast cells (ZurNieden et al., 2003; Philips et al., 2001), and neural precursors having different phenotypes (Fraichard et al., 1995; Plachta et al., 2004; Glaser and Brustle, 2005).

The reproducibility of such approaches is sometimes difficult and does not make it possible to obtain satisfactory cell enrichments for industrial applications, such as replication of specific viruses or the production of molecules of interest. Moreover, the processes include numerous steps, involving the use of very specific culture media and numerous cell manipulation steps.

The approaches described above make it possible to induce stem cell differentiation; since the populations obtained are heterogeneous, it is then necessary to select and isolate the cells having the desired phenotype; for this, a number of techniques are known by a person skilled in the art. One may cite, in a non-exhaustive manner, cell sorting with a surface sorter after marking with specific surface antibodies, and magnetic enrichment or depletion.

Another approach consists of using enrichment protocols that use positive genetic selection processes. For this, the expression of a gene for selection of a drug (neomycin, hygromycin, zeomycin, blasticidine) or a phenotype marker enabling the physical sorting of a cell expressing it (fluorescent proteins such as wild-type or modified GFP, beta-galactosidase, alcohol dehydrogenase) is placed under the dependence of a tissue-specific promoter. Among the promoters used, it is possible to cite those of Myosin chains (Muller et al., 2000), those of Myf5 or MyoD myogenesis inducing genes, those of Nestin (Keyoung et al., 2001), Hb9 (SinghRoy et al., 2005), specific astrocyte promoters such as GFAP (Benveniste et al., 2005) or from other neural types such as oligodendrocytes with CNP genes (cyclic 3′ phosphodiesterase) (Glaser et al., 2005; Schmandt et al., 2005).

Another approach consists of overexpressing the cDNA of interest capable of inducing differentiation in a given lineage, such as, for example, with the cDNA of the myoblastic lineage (Pax3, MyoD, Myf5, Myogenin, Mft-4 genes, etc.). This overexpression can be achieved with randomly inserted overexpression vectors, but also with a knock-in strategy in a given locus of actin or in the specific locus of the lineage, or with viral and retroviral vectors.

At present, there is no method making it possible to obtain a population of homogeneous in vitro differentiated cells, without subsequently performing a selection step. This is why the inventors have developed a new technique making it possible to obtain a differentiated cell population with homogeneous morphological, biochemical and functional characteristics, that does not require the additional selection step. According to the invention, it is possible to obtain a morphological, phenotypic and molecular modification of 80 to 100% of the cells of an induced clone, i.e. a clone comprising differentiated cells.

In mammals, a plurality of genes controlling the pluripotency and proliferation capacities of stem cells have been identified. In particular, the gene Oct4 (Nichols et al., 1998) is a key gene expressed only in pluripotent cells in vivo. In vitro, Oct4 is expressed in mouse and primate embryonic stem cells, as well as in certain tumor cell lines. The expression level of Oct4 in murine embryonic stem cells appears to control the outcome of these stem cells (Niwa et al., 2000).

The Oct4 gene belongs to the POU family of transcription factors with an homeodomain; the protein produced from the gene has a nuclear localization and can bind directly to the target gene regulation elements via its DNA-binding domain.

From a phylogenetic perspective, it has been unlikely for a gene of equivalent function to exist in non-mammal species. In birds, such a gene could not be identified in earlier studies (Sooden-Karamath & Gibbins, 2001). Moreover, no homology at the genome level has been reported.

In mammals, the Nanog (Chambers et al. 2003: Mitsui et al., 2003; Hart et al; 2004) and Eomes (Russ et al., 2000, Ginis et al., 2004) genes have also been identified as playing a key role in maintaining the pluripotency of stem cells.

This invention proposes a process for preparing differentiated avian cells from avian stem cells cultivated in an appropriate culture medium, characterized in that it includes a step of inducing the differentiation of the stem cells by inhibiting the expression or activity of a gene expressed in said stem cells, selected from the genes 1P06, Nanog and Eomes.

The advantage of this process is that the differentiated cells obtained are homogeneous. The clones obtained have no longer the characteristic morphology of a stem cell clone, but a differentiated morphology. These clones have no longer the proliferation characteristics of parental cells and the cellularity remains constant after induction.

This induction of the cell differentiation is both fast and irreversible.

Preferably, the avian cells come from a bird belonging to the Galliformes order, in particular a chicken or a quail, and more preferably of the Gallus gallus species.

By “stem cells”, a person skilled in the art understands that the cells have the following characteristics:
the capacity to proliferate by self-renewal in vitro in the presence of growth factors well known in the literature, and over significant time periods;
from a morphological perspective, a stem cell is characterized by a high nucleocytoplasmic ratio, a relative size of 10 to 15 mm with a core of 5 to 10 mm, has various intrinsic biochemical activities such as those of alkaline phosphatase and telomerase, and is recognized by various specific antibodies as described in patents FR 94/12598 and FR 00/06029. Another particularity of avian embryonic stem cells is their close membrane contact when they proliferate;
the cells are pluripotent, which means that they can produce a plurality of cell types; in particular, the avian stem cells of the invention can be differentiated into ectodermic, mesodermic and endodermic derivative cells.

By “differentiated cells”, a person skilled in the art understands that the cells proliferate more or less entirely, that they have a lower nucleocytoplasmic ration, a generally larger size (> 15-20 mm or more for certain specific cell types such as osteoclasts, mature adipocytes, differentiated muscle cells, certain macrophages, etc.), with a less compact morphology, and that they have lost the biochemical markers of the non-induced stem cells.

In particular, stem cells can be differentiated into epithelial cells, characterized by the presence of markers such as cytokeratins 8, 18 and 19 or keratin 16 for keratinocytes, and certain specific mucins, cadherins and integrins. Depending on the origin of the epithelial cells considered, various associations of markers will be characteristics of the origin and the degree of differentiation of the epithelial cells, in particular in neoplasic processes (Barak et al., 2004).

According to another aspect of the invention, the stem cells can be differentiated into precursor and differentiated hematopoietic cells, characterized inter alia by the presence of membrane markers, known in mice and humans by the CD (Cluster of Differentiation) nomenclature and presented by Lai et al. in a review published in the Journal of Immunology (1998). Other non-membrane markers can also be used, in particular specific transcription factors.

By “gene expression or activity inhibition”, a person skilled in the art understands that all of the usual means making it possible to control gene expression are capable of being used, in particular transcription or translation inhibitors. In particular, the use of interfering RNA makes it possible to specifically inhibit the expression of genes of interest.

According to a specific aspect of the invention, the gene expression or activity inhibition is performed conditionally. Thus, the differentiation of cells can be initiated at a time desired by a person skilled in the art.

According to a specific aspect of the invention, the gene expression or activity inhibition is performed with at least one interfering RNA.

The interfering RNA molecules are used to specifically target messenger RNA (mRNA): the interfering RNA hybridizes with the mRNA and consequently inhibits the translation of the corresponding protein, either by simple steric hindrance, or by promoting mRNA cleavage.

This strategy of gene expression inhibition has been applied to murine embryonic stem cells both in vitro and in vivo (Grabarek et al., 2003; Kunath et al., 2003; Lickert et al., 2005). The use of RNAi in ovo in chicken is possible as demonstrated in (Nakamura et al., 2004).

The interfering RNA can be chosen from a plurality of forms, such as: antisense RNA, double-stranded RNA, “small interfering” RNA, “Small Hairpin” RNA or ribosomal RNA.

The siRNA, for “Small Interfering RNA” are short sequences of around 15 to 30 base pairs (bp), preferably 19 to 25 bp. They include a first strand and an additional strand identical to the targeted region of the RNA of the target gene. By “siRNA”, we mean the synthetic forms of a double-stranded RNA.

The shRNA for “Small Hairpin RNA” are double-stranded RNA produced by a cloned sequence in an expression vector, coding for a RNA molecule that will adopt a hairpin shape after cleavage by the Dicer and loquacious complex (Du et al; 2005).

The design and preparation of interfering RNA and their use in the transfection of cells in vivo and in vitro are well known and widely described in numerous publications, such as: U.S. Pat. No. 6,506,559, US 2003/0056235, WO 99/32619, WO 01/75164, WO 02/44321, US 2002/0086356, WO 00/44895, WO 02/055692, WO 02/055693, WO 03/033700, WO 03/035082, WO 03/035083, WO 03/035868, WO 03/035869, WO 03/035870, WO 03/035876, WO 01/68836, US 2002/0162126, WO 03/020931, WO 03/008573, WO 01/70949, WO 99/49029, U.S. Pat. No. 6,573,099, WO 2005/00320, WO 2004/035615, WO 2004/019973, WO 2004/015107, http://www.atugen.com/sirnatechnology.htm, http://www.alnylam.com/science-technology/index.asp, http://www.protocol-online.org/prot/Research_Tools/Online_Tools/SiRNA_Design/, http://www.hgmp.mrc.ac.uk/Software/EMBOSS/Apps/sirna.html, http://www.rockefeller.edu/labheads/tuschl/sirna.html, http://www.upstate.com/browse/categories/siRNA.q.

The siRNA can be designed and prepared by using suitable software available online, such as:
“siSearch Program” http://sonnhammer.cgb.ki.se/siSearch/siSearch_{—}1.6.html (“Improved and automated prediction of effective siRNA”, Chalk A M, Wahlesdelt C, and Sonnhammer ELL, Biochemical and Biophysical Research Communications, 2004).
“SiDirect” http://design.rnai.jp/sidirect/index.php (Direct: highly effective, target-specific siRNA design software for mammalian RNA interference, Yuki Naito et al, Nucleic Acids Res, Vol. 32, No. Web Server issue© Oxford University Press 2004).
“siRNA design tool” of Whitehead Institute of Biomedical Research at MIT http://jura.wi.mit.edu/pubint/http://iona.wi.mit.edu/siRNAext/
siRNA Wizard™ of Invitrogen http://www.sirnawizard.com/,
“siRNA Target Finder” of Ambion http://www.ambion.com/techlib/misc/siRNA_finder.html,
https://www.genscript.com/ssl-bin/app/rnai
http://www.promega.com/siRNADesigner/default.htm
http://bioweb.pasteur.fr/seqanal/interfaces/sirna.html
Other programs are referenced on the site http://web.mit.edu/mmcmanus/www/home1.2files/siRNAs.htm, such as http://athena.bioc.uvic.ca/cgi-bin/emboss.pl?_action=input&_app=sirna.

The tools for preparing siRNA and the transfection of cells are available to the public, such as, for example, the siRNA vectors sold by the Invitrogen company (http://www.invitrogen.com).

According to the interfering RNA sequences selected by a person skilled in the art, various inhibition levels can be obtained, making it possible to modulate the inhibiting effect as desired. Preferably, the interfering RNAs are prepared and selected to obtain at least 50% of inhibition of the expression of the target gene in a cell, and even at least 75%, 90%, 95% and up to more than 99% of inhibition. A person skilled in the art would known how to select the most effective RNAi using simple routine tasks, for example by analyzing their capacity to induce stem cell differentiation (see FIGS. 1 and 3).

According to a preferred embodiment of the invention, the interfering RNA specifically inhibits the expression of the gene 1P06.

In particular, the nucleic acid molecule coding for the interfering RNA can have a sequence as presented in the sequence list under the heading SEQ ID N^{o} 3.

According to another preferred embodiment of the invention, the interfering RNA specifically inhibits the expression of the Nanog gene.

In particular, the nucleic acid molecule coding for the interfering RNA can have the sequence SEQ ID N^{o}4 or SEQ ID N^{o}5.

According to another preferred embodiment of the invention, the interfering RNA specifically inhibits the expression of the Eomes gene.

In particular, the nucleic acid molecule coding for the interfering RNA can have one of the sequences as represented by SEQ ID N^{o}6, N^{o}7, N^{o}8, or N^{o}9. The molecule with which the best expression inhibition is obtained is coded by sequence SEQ ID N^{o} 4.

A standard technique for inhibiting the expression of a gene with an interfering RNA consists of introducing a double-stranded RNA into the cell; however, as this dsRNA will quickly be degraded, the gene inhibition will be limited over time. This limitation led the inventors to look for a system making it possible to produce interfering RNA “on request” in the target cell.

This invention relates to expression vectors including a nucleic acid molecule coding for an interfering RNA, placed under the control of a promoter enabling the expression of said interfering RNA in a host cell. The host cell will preferably be an avian stem cell maintained in an in vitro culture.

Said vectors preferably include a promoter, translation initiation and termination signals, as well as suitable transcription regulation regions.

According to a specific embodiment of the invention, the promoter of the expression vector that controls the expression of the interfering RNA is an inducible promoter.

The advantage of an inducible expression system is that it is possible to control the time of presence of the interfering RNA, and therefore the inhibition of the target gene. Indeed, certain genes, essential for cell survival, cannot be inhibited for too long because it would induce cell death; other genes must be inhibited for a longer time before it is possible to observe effects of this inhibition. The control of the interfering RNA expression in the host cell makes it possible to overcome these two constraints.

A person skilled in the art knows various types of inducible transcriptional promoters, such as, for example, those of the lactose operon, which was the first identified and analyzed. One can also cite the tet off/tet on system based on a promoter derived from the tetracycline resistance operon of E. coli (Gossen et al., 1992).

The activity of a promoter can be induced either by activating an inactive promoter, or by removing the inhibition on a constitutionally active promoter. Each transcription inhibiting or activating factor can itself be inducible depending on the cell environment.

For example, if the promoter has a domain for binding to transcription inhibiting factors, the removal of the inhibition will be done by trapping these factors, which can no longer bind to the regulatory sequences of the promoter; the promoter will then be “released” from the transcription inhibiting factors.

According to another example, the promoter is constitutionally active, but is inhibited by the presence of a cassette inserted into its locus; the cassette is placed so that the regulatory elements of the promoter can no longer cooperate with one another because they are too far apart; the removal of the inhibition is then done by excising the cassette, in particular by use of sequences recognized by a recombinase, placed at each end of the cassette. The cassette can include a long DNA “buffer” sequence serving solely to create the steric hindrance (Tiscornia et al., 2004), but it can also include “marker” genes such as, for example, antibiotic resistant genes, or fluorescence genes such as GFP (Green Fluorescence Protein). The presence of this selection cassette also makes it possible to maintain the locus in an active chromatin configuration, which limits the “silencing” mechanisms of the sequences, in particular in the stem cells.

To excise the cassette, two systems are commonly used: the Cre-Lox system and the FLP-FRT system. The Cre-Lox system is based on the capacity of an enzyme, Cre recombinase, to suppress any DNA fragment localized between specific DNA sequences: the loxP sequences. The FLP-FRT system is based on the activity of FLP recombinase, which suppresses the DNA fragments localized between two FRT sequences. The sites of Lox P and FRT recognition and the corresponding Cre and FLP recombinases are well known to a person skilled in the art, in particular described in Kilby, et al.; 1993, Sauer et Henderson, 1988, Gu et al.; 1994, Cohen-Tannoudji et Babinet; 1998, Shibata et al.; 1997, Schlake et Bode 1994.

In particular, an inducible promoter can contain a lox—promoter cassette of Thymidine kinase (TK)-Neomycin^{R}-lox, the presence of which inhibits its functioning (Coumoul et al., 2004). The promoter becomes functional only after excision by the CRE recombinase of this cassette. The CRE recombinase used itself has an activity dependent on the presence of tamoxifen in the culture medium (Metzger et al., 1999). Thus, by adding tamoxifen to the cell culture medium, the recombinase becomes active and excises the inhibiting sequence of the promoter, thus making the promoter functional, thus enabling the expression of the interfering RNA.

Among the promoters of interest, dependent pol III promoters are preferably chosen, such as the promoter U6 or H1, well known to a person skilled in the art for their use in the expression of interfering RNA.

The expression vector according to the invention can be integrated in a host cell, in particular an avian cell maintained in culture in vitro. The cells are then said to be “transformed”. To do this, it is possible to use self-replicating vectors in the host cell, or integrative vectors, which will enable the integration of the exogenous nucleic acid into the DNA of the host cell.

Among the self-replicating systems, plasmid or viral systems are preferably used, and the viral vectors can in particular be adenoviruses (Perricaudet et al., 1992), retroviruses, lentiviruses, poxviruses or herpes viruses (Epstein et al., 1992). A person skilled in the art knows the technologies that can be used for each of these systems.

When it is desirable to integrate the sequence into the chromosomes of the host cell, it is possible to use, for example, plasmid or viral systems; such viruses are, for example, retroviruses (Temin, 1986), or AAV (Carter, 1993).

Among the non-viral vectors, naked polynucleotides are preferred, such as naked DNA or naked RNA, according to the technique developed by the VICAL company.

In avian cells, avian adenoviruses or retroviruses, poxiviruses or naked DNA introduced by transfection or electroporation will preferably be used.

Such vectors are prepared according to methods commonly used by a person skilled in the art, and the clones resulting therefrom can be introduced into a host cell by standard methods, such as lipofection, electroporation, heat shock, transformation after chemical permeabilization of the membrane or cell fusion.

According to another aspect of the invention, the process is characterized by the fact that the cells are subjected to at least one step of specific gene activation or inhibition, with the goal of obtaining a specific differentiation of said cells.

In particular, the use of certain promoters and coding phases makes it possible to control the expression of genes that themselves induce the terminal differentiation of cells in defined pathways. These genes code in particular for transcription factors. Among these genes, one may cite regulators of the myogenic lineage (‘MRF’) such as Myf5, MyoD, Mrf4, those involved in the determinism of the adipocyte line such as the PPARg gene, in neural determinism such as Sox1 and Nestine, and in the determinism of the hematopoietic pathway such as Gata2 and Gata3.

This step of inhibiting or expressing gene activity can be either simultaneous to the step of inhibiting genes chosen from 1P06, Nanog or Eomes, or delayed in time, preferably following the inhibition induction by a few hours. This delay enables the cell to initiate the terminal differentiation program sequentially, and thus to better reproduce the differentiation kinetics observed in vivo.

According to another aspect of the method of the invention, the avian stem cells of the invention are cultivated under appropriate conditions, in particular in the presence of certain growth factors and inducers, in order to obtain a specific differentiation of said cells.

Among these factors, it is possible to cite growth factors of the BMP family, the FGF family or the WNT family, of which the very complex relative roles in the control of the mesodermic and ectodermic pathways are beginning to be described in the mouse, the Xenopus and the chicken. These factors will preferably be used in a concentration range on the order of 1 to 10 ng/ml, in particular for BMP-4 and BMP-7. Many other factors are involved in the inductions of ecto and mesodermic pathways.

The process according to the invention can also include a step of selecting and destroying cells not induced by differentiation.

The disappearance of non-induced clones can be achieved concomitantly to the induction, by a negative selection using a vector expressing a toxic gene (thymidine kinase, diphtheria toxin), placed under the control of an active promoter only in stem cells, for example the promoter of the Ens1 gene or of the 1P06 gene described below. Thus, only the cells with the stem cell characteristics will disappear.

The invention also relates to differentiated avian cells capable of being obtained by the process according to the invention.

The invention also relates to a process for preparing an antiviral vaccine, characterized in that it includes a step of contacting a virus with avian stem cells that have been subjected to the process according to the invention.

Twenty-four to seventy-two hours after the induction of the stem cell differentiation, the cells are exposed to the virus of interest in an appropriate culture medium. It is necessary to place the virus in contact with the cells while they are in their induction phase in order to have a residual division enabling the integration of the virus and its production, even if the cells induced divide only slightly with respect to the proliferating cells. The doubling time increases from around 12 to 15 hours to more than 24 to 36 hours in the early induction phase in order to arrive at a cell that no longer divides after 96 hours from the start of the induction. The M.O.I, necessary for infection are calculated based on the number of cells obtained after 24 to 48 hours from the start of the induction.

The stem cells and their differentiated derivatives can serve as a support for the replication of various viruses according to the specific tropism of said viruses. This strategy can be developed successfully with stem cells in view of their high phenotypic plasticity. The adaptation of many viruses to uniform laboratory cell substrates (such as cells of lines BHK-21 and CV-1 for example) modifies the production efficiencies of the viruses by affecting their replicative cycle. The mechanisms controlling these restrictions are beginning to be elucidated (Wang & Shenk, 2005).

The invention also relates to a nucleic acid molecule coding for an interfering RNA as defined above in the invention.

The invention also relates to an expression vector including a nucleic acid molecule coding for an interfering RNA according to the invention, placed under the control of a promoter enabling the expression of said interfering RNA in a host cell, preferably an avian cell maintained in an in vitro culture.

According to a preferred aspect of the invention, this promoter can be induced.

The invention also relates to avian stem cells transformed with an expression vector as described above.

The invention also relates to a nucleic acid sequence coding for a polypeptide of which the protein sequence has at least 68% identity with the amino acid sequence according to SEQ ID N^{o}1 (see sequence listing).

Moreover, this invention also comprises a polypeptide whose protein sequence has at least 68% identity with the amino acid sequence according to SEQ ID N^{o}1.

The BLAST (Basic Local Alignment Search Tool) software program was used to compare this new protein sequence with those already known and indexed in the databases. Of all of the proteins already known, the one with the highest level of identity has a protein sequence 67% identical to sequence SEQ ID N^{o}1.

The invention also relates to a nucleic acid sequence having at least 94% identity with SEQ ID N^{o}2.

Just as for the protein sequence, SEQ ID N^{o}2 was compared with sequences already known and indexed using the BLAST software; the highest percentage of identity was 93%.

The invention also relates to a cloning and/or expression vector into which a nucleic acid molecule having one of the sequences as described above is inserted.

The invention also relates to a nucleic acid sequence having at least 90% identity with the nucleotide sequence according to SEQ ID N^{o}11, which corresponds to the promoter region of the Nanog gene, i.e. the 12112 base pairs preceding the ATG codon beginning the coding sequence of the chicken Nanog gene.

This promoter region of the Nanog gene contains elements regulating this gene, and more specifically binding sites for transcription regulation factors. As this gene is expressed only in stem cells, the study of mechanisms regulating its expression is particularly important for determining the conditions in which the stem cells remain undifferentiated. A person skilled in the art can identify the regulatory factors capable of binding to this promoter sequence, as well as their precise sites at which they bind to the DNA, whether they are already known or are newly identified. For additional information, see (van Steensel B., 2005, Nat Genetics), (Siggia E D., 2005, Curr Opin Genet Dev) and (Pavesi G, Mauri G, Pesole G, 2004, Brief Bioinform). In particular, regulatory sequences already known can be identified by conducting research using the TRANSFAC, TRRD and COMPEL databases, which index gene expression regulation sequences. These databases can be accessed at the following addresses: http://trarisfac.gbf.de/TRANSFAC or http://www.bionet.nsc.ru/TRRD.

The invention also relates to a cloning and/or expression vector into which a nucleic acid molecule having the sequence as defined above is inserted.

The invention also relates to a prokaryotic or eukaryotic cell having integrated one of the cloning and/or expression vectors as described above.

The invention also relates to a process for culturing stem cells, and more specifically avian stem cells, in which the cells are cultivated in a culture medium including no more than around 1% serum. This process is characterized in that stem cells in which the Nanog gene is overexpressed are cultivated. This overexpression can be obtained by any means known to a person skilled in the art, and in particular by modifying cells with a so-called “strong” promoter controlling the expression of the endogenous Nanog gene, or by introducing a vector overexpressing the Nanog gene. This is in particular performed by introducing a vector overexpressing the chicken Nanog gene into the stem cells.

This gene indeed has the characteristic of conferring on the cells an “independence” with respect to the growth factors contained in the serum. The cells thus transformed can be cultivated and transferred for as long as the initial stem cells, in the medium containing 1% serum.

This process opens a wide field of industrial applications: on the one hand, this process is economical (usually, no more than 1% serum is used rather than 10%); on the other hand, it is advantageous to be capable of cultivating cells with a minimal amount of serum when one wants to determine the specific effect of growth factors that may be present in the serum.

The examples below illustrate the invention, and should not be considered to limit it.

### DESCRIPTION OF THE FIGURES

FIG. 1: The inhibition of the expression of the 1P06 gene by specific RNAi induces a drastic loss in the proliferation of CESC cells and induces their differentiation. This effect is measured on the morphology of clones of which the differentiation has been induced by hydroxytamoxifen.

FIG. 2: Molecular analysis of differentiated clones obtained by inhibition of the expression of the 1P06 gene by RNAi-1P06-2. The transcriptomic contents of the differentiated or proliferative clones is analyzed by real-time PCR.

FIG. 3: The inhibition of the expression of the Nanog gene by specific RNAi induces the differentiation of CESC cells and therefore stops the cell proliferation. This effect is measured on the morphology of the clones whose differentiation has been induced by hydroxytamoxifen.

FIG. 4: Inhibition of the expression of 1P06 and Nanog genes by a specific interfering RNA:

A—Marking of cells induced into differentiation by the anti-SSEA-1 antibody

B—Kinetics of the stopping of proliferation induced by interfering RNA

C—Molecular analysis of the expression of differentiation marker genes in the cells after inducing this differentiation

FIG. 5: Phylogenetic tree of the various members of the POU multifamily, including orthologs and paralogs of the 1P06 G. gallus gene.

FIG. 6: Percentages of homology of the 1P06 G. gallus gene with its orthologs of different species.

FIG. 7: Percentages of homology of the 1P06 gene with other factors of the POU family in the G. gallus species.

FIG. 8: Localization of the GFP-1P06 fusion protein in proliferating CESC cells (N: nuclear; C: cytoplasmic; N+C: nuclear and cytoplasmic).

FIG. 9: Impact of the overexpression of the GFP-1P06 fusion protein in proliferating CESC cells. The level of structuration of the chromatin is determined.

### EXAMPLES

### Example 1

### Induction of Avian Stem Cell Differentiation by Inhibition of the 1P06 Gene by Means of an Interfering RNA

### a) Construction of the Expression Vector Coding for the Interfering RNA

The plasmid pFLΔNeo is obtained by insertion of the fragment mU6DneoD of 2 kb, amplified by PCR in the SmaI/HindIII sites of the pBSK (Stratagene). This fragment mU6DneoD is amplified with the primer oligonucleotides m-U6-smaI-S and m-U6-HindIII-AS using the matrix mU6D-Neo-DApaIDXhoI, provided to us by Dr. Sheng, NIH Bethesda, and described in the article (Coumoul et al., 2004).

Once designed using the software provided at the sites http://www.proligo.com; http://www.qiagen.com, http://www.ambion.com, 100 pM of each RNAi oligonucleotide are hybridized to obtain the double-stranded fragment by slow renaturation at room temperature in the presence of 1 mM MgCl₂ after denaturation by heating at 95° C. for 5 min. The resulting double-stranded oligonucleotide is cloned directly in the HindIII/XhoI sites of the pFLDNeo vector prepared by digestion with these same enzymes. The screening is validated by the disappearance of the Hindi site and by a systematic sequencing of these positive plasmids. For each target RNA ‘X’, the pFLDNeo X-RNAi vector is thus obtained. The sequence of the different RNAi used is presented in the sequence listing before transfection, the vectors are linearized by AhdI, a single site present in the ampicillin-resistant gene.

The expression vector of CRE-ERT2-hygro recombinase differentiates from the commercial vector pCI-Neo (Promega) after a plurality of steps. The hygromycin resistance cassette is obtained from the commercial vector pIRES-Hygro (Clontech) by PCR amplification using the oligonucleotide pair NruI-hygroS and BamHI-hygro AS. The resulting fragment is directly cloned into NruI-BamHI in the pCI-Neo vector in which the neomycin resistance cassette had earlier been removed by KpnI-BamHI digestion. The resulting plasmid is the pCI FLHygro plasmid. The coding phase of the CRE-ERT2 recombinase is amplified on the basis of the pCRE-ERT2 plasmid, provided by Pr. P. Chambon, IGMC, Strasbourg, and described in the publication Feil et al., 1997 by using the oligonucleotide pair CRE-ERT2-SalI S and CRE-ERT2-smaI AS. The resulting amplified fragment is directly cloned in the receiving vector pCI FL-Hygro prepared by SalI-SmaI digestion. The cloning is directional and results in the pCRE-ERT2-Hygro vector. The set of coding phases is sequenced so as to verify the completeness of the amplifications and enable proper activity of the recombinase.

### b) Introduction of the Expression Vector Comprising the Interfering RNA in the Avian Stem Cells

The CESC cells are obtained, amplified and maintained in vitro as described in Pain et al., 1996 and the applications FR N^{o}94/12598, FR N^{o} 00/06029. For transfections, 0.5 to 1×10⁶ cells are transfected using liposomes (Fugene 6, Roche) as described earlier (Pain et al., 1999, Patent applications FR N^{o} 00/06029 and FR N^{o} 01/15111) using 2 to 10 mg of the different linearized vectors. The first step consists of transfecting the expression vectors pFLDNeo X-RNAi. The neomycin selection (from 100 to 250 mg/ml) is applied for 7 days. The resistant clones obtained are counted and can be collected, amplified and frozen individually for future use after amplification. They integrated the expression vector of the RNAi, but do not express it. These clones are then dissociated and subjected to a new transfection by the expression vector of pCRE-ERT2-Hygro recombinase. The cells are selected in the presence of hygromycin (from 25 to 75 mg/ml) with a maintained presence of a neomycin selection (from 50 to 200 mg/ml). After 7 days, the clones obtained are again counted, collected and amplified individually.

### c) Conditional Expression of the Interfering RNA in the Stem Cells

The vector pFLD Neo XRNAi makes it possible to control the expression of RNAi by the presence of a lox-TK-Neo-lox sequence, in the murine U6 promoter, which sequence renders the functioning of said promoter conditional (Coumoul et al., 2004). The promoter becomes functional after excision by CRE recombinase of this insertion. We used in particular a CRE-ERT2 recombinase, of which the activity is dependent on the presence of tamoxifen in the culture medium (Metzger et al., 1999).

On the clones obtained after the two transfection steps described, the addition of 1 mM of 4-hydroxytamoxifen to the culture medium for 96 hours induces the excision of the neomycin resistance cassette, contained by the loxP sites, and enables the production of the target RNAi.

### d) Morphological Characterization of the Differentiated Cells Obtained

After induction by 4-hydroxytamoxifen of the expression of CRE recombinase, the morphology of the cells obtained is analyzed by direct microscopic observation and recording using a CCD CoolSNAP Camera (Photometrix).

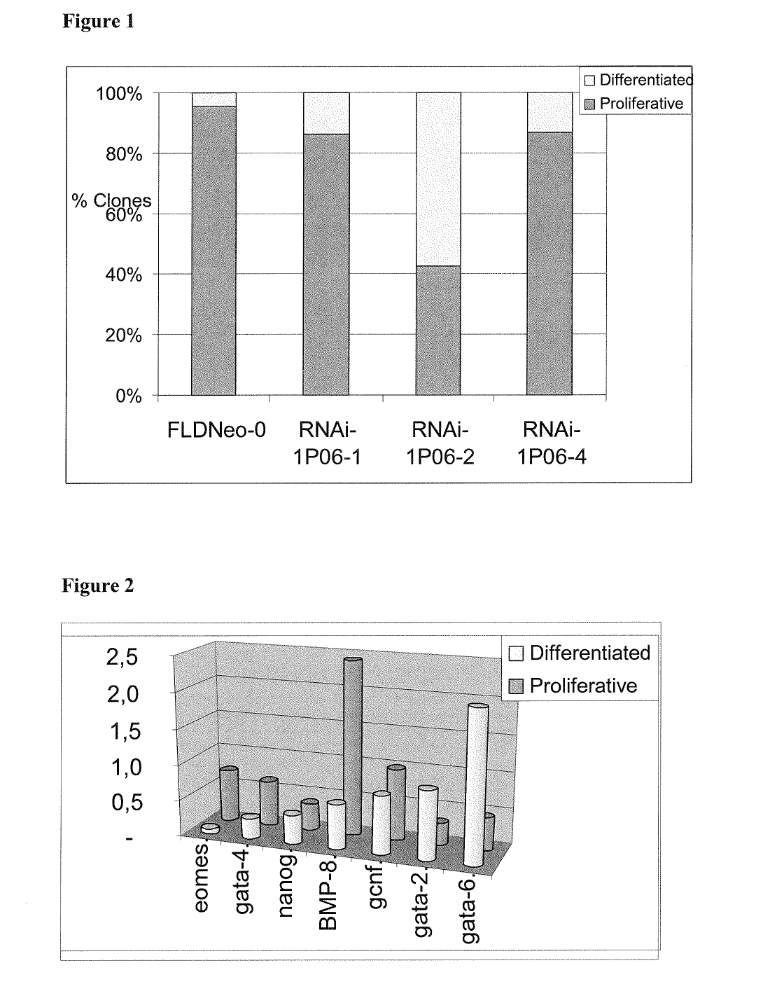

The clones are counted 4 days (96 hours) after induction by the 4-hydroxytamoxifen; the results obtained are shown in FIG. 1. The total number of clones obtained in 3 independent experiments is respectively n= 45 for pFLDNeo-0 (empty control vector), n=59 for pFLDNeo-1, n= 61 for pFLDNeo-2 and n= 76 for pFLANeo-4.

FIG. 1 shows the percentage of clones of which the differentiation was induced and the percentage of clones that continue to proliferate. Only the RNAi-2 makes it possible to obtain the reverse induction phenomenon of the empty control vector and RNAi-1 and -4, for which no major phenotype change is observed, as shown by the number of undifferentiated clones.

Thus, even in the presence of the growth factors and cytokines necessary for proliferation of undifferentiated cells, one observes in the cells expressing RNAi-2 a fast and drastic change in the morphology of the cells in the clones. The proliferative capacity of these clones is lost, and they become proportionally smaller with respect to the non-induced clones. These non-induced clones can result from a non-excision of the lox cassette by recombinase, and therefore a non-expression of the RNAi.

The induced cells have a flat morphology, with a larger size than the non-induced parental cells. Scanning microscopy observations also show a larger spread of the induced cells.

### e) Molecular Characterization of Induced Cells

To characterize, at the molecular level, the clones induced by the expression of the RNAi inhibiting the expression of the 1P06 gene, differentiated and undifferentiated clones were collected and analyzed. The results obtained are presented in FIG. 2. The expression level of various marker genes is shown. Level 1 represents the level observed in clones having integrated the empty vector pFLDNeo-0.

The analysis of the transcriptomic contents of these clones by real-time RT-PCR approaches indicates that the overall expression level of the 1P06 gene is less important in these clones with respect to the parental cells (result not shown). The expression levels of the Nanog, Eomes and Gcnf genes are reduced. Conversely, the expression of the Gata6 gene is increased, suggesting an induction in a differentiation pathway equivalent to the primitive endoderm.

### Example 2

### Induction of Avian Stem Cell Differentiation by Inhibiting the Expression of the Nanog Gene by Means of an Interfering RNA

By conducting experiments similar to those conducted on the 1P06 gene, with RNAi expression vectors directed against the Nanog gene, an inhibition of proliferation and an induction of the differentiation of selected clones are also observed.

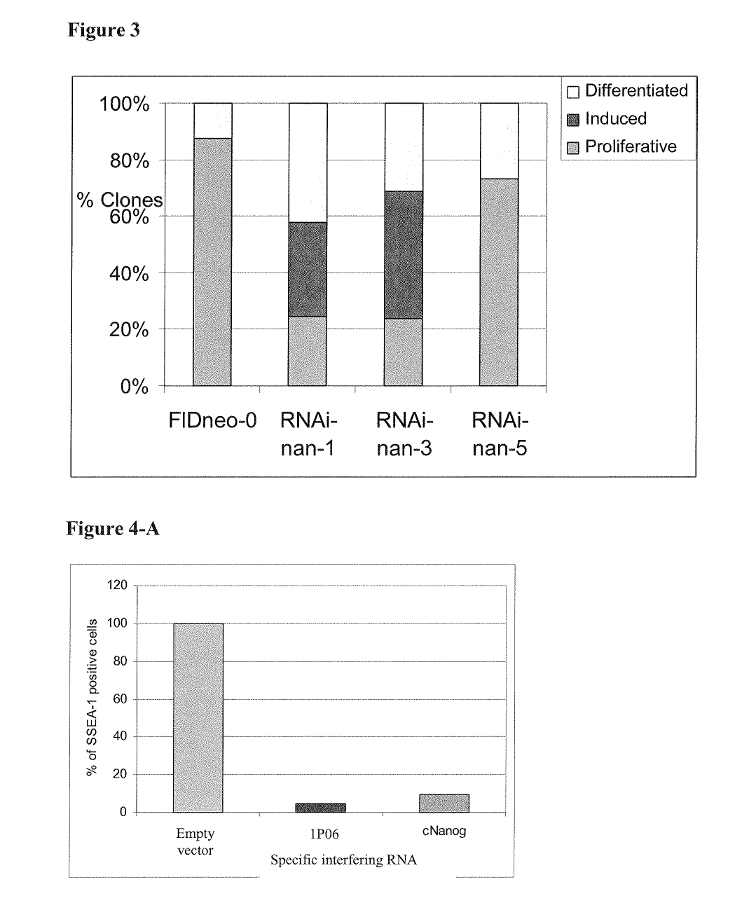

The clones are counted 4 days (96 hours) after the induction of CRE recombinase by the addition of 4-hydroxytamoxifen. The total number of clones obtained is respectively n= 5 for pFLDNeo-0 (empty controlled vector), n= 21 for pFLDNeo-nan-1, n= 37 for pFLΔNeo-nan-3, n= 15 for pFLDNeo-nan-5. The results obtained are shown in FIG. 3.

FIG. 3 shows the percentage of clones, of which the differentiation was induced (induced or differentiated clones) and the percentage of clones continuing to proliferate. By contrast with the empty vector, the RNAi-nan1 and RNAi-nan3 are capable of inducing a differentiation of most of the cells: less than 30% of the clones continue to proliferate. RNAi-nan-5 also induces this differentiation, to a lesser extent.

### Example 3

### Induction of Differentiation By Inhibiting the 1P06 and Nanog Genes in Parallel

In these experiments, the shRNA used were the following:

RNAi-2 for inactivation of 1P06, and RNAi-nan1 for inactivation of Nanog.

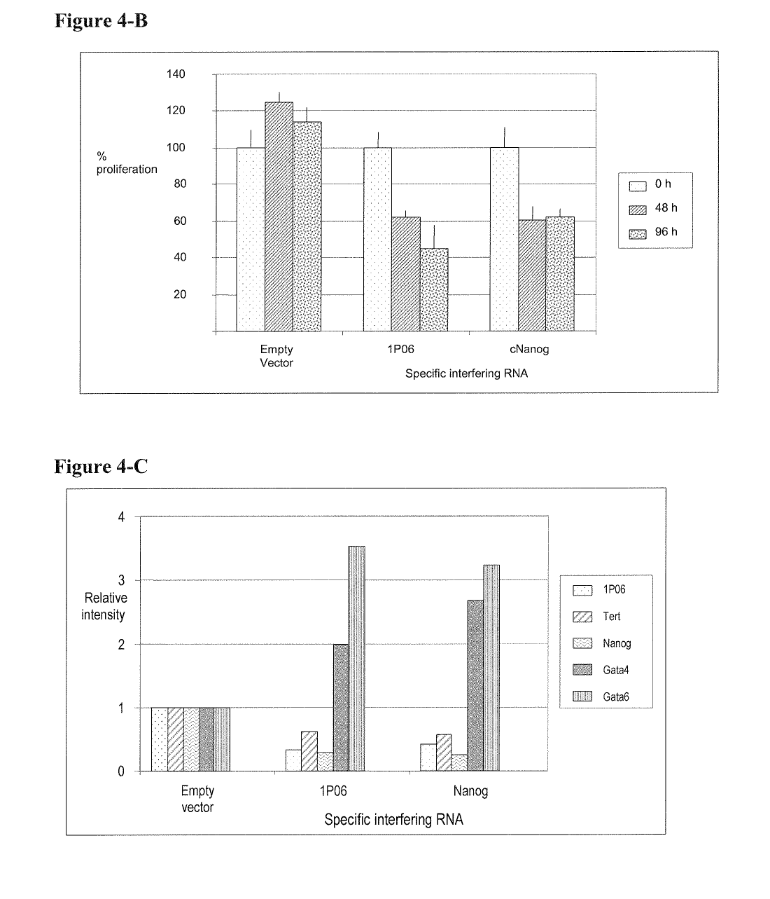

The differentiation of avian stem cells, after induction by inhibition of the expression of 1P06 and Nanog genes, was evaluated by the following measures:
percentage of cells positive for marking by anti-SSEA-1 antibodies (FIG. 4A)
percentage of proliferation of clones obtained over 48 and 96 hours (FIG. 4B)
relative expression of “marker” genes: 1P06, Tert, Nanog, Gata4, Gata6 (FIG. 4C).

FIG. 4A shows that the large majority of cells obtained no longer express the SSEA-1 antigen (Stage-Specific Embryonic Antigen-1), of which the expression is characteristic of stem cells (100% positive cells when the transfection is performed with an empty vector).

FIG. 4B shows the timeline of events, and in particular the fact that the stop of clone proliferation is obtained quickly.

The clones are counted by direct microscopic observation, as described above, at 48 and 96 hours after induction by 4-hydroxytamoxifen; the figure shows the relative percentage of clones of which the differentiation was induced or which continue to proliferate: after 48 hours, only 60% of the clones continue to proliferate; this is the maximal effect obtained by inhibiting the expression of the Nanog gene; after 96 hours of inhibition of the 1P06 gene, no more than 40% of proliferating clones are observed.

FIG. 4C shows the molecular characterization of cells induced into differentiation by inhibition of the expression of 1P06 and Nanog genes.

The expression of the various “marker” genes is normalized with respect to the expression level “1” observed in the clones having integrated the empty vector pFLDNeo-0.

The analysis of the transcriptomic contents of these clones by real-time RT-PCR approaches indicates that the overall expression level of the 1P06 gene is lower after differentiation. The expression levels of the Tert and Nanog genes are reduced. Conversely, the Gata-4 and Gata-6 genes have an increased expression, suggesting an induction into a differentiation pathway equivalent to the primitive endoderm.

### Example 4

### Cloning of the 1P06 Gene

### Oligonucleotides and Sequencing

All of the oligonucleotides were designed using the Primer 3 software program (http://frodo.wi.mit.edu/cgi-bin/primer3/primer3_www.cgi, then synthesized by the Proligo company. The sequences of the various genes tested are identified in the TIGR index (http://www.tigr.org/tigr-scripts/tgi/T_index.cgi?species=g_{—}gallus) or found directly at the chicken genome assembly site (http://www.ensembl.org/Gallus_gallus/) or obtained directly from newly identified sequences.

### Extraction of RNA

The total RNAs of the undifferentiated cells in proliferation or the CESC cells induced by differentiation in embryoid bodies for 2 days are extracted using the RNAEasy mini kit (Quiagen, ref 74104). The total RNA of the clones are extracted using the RNA Easy micro kit (Qiagen ref 74004). The quantity and quality of the RNA are measured by the BioAnalyser 2100 (Agilent Biotechnologies).

### Subtractive Liquid Hybridation

After a step of reverse transcription, the cDNA are subjected to digestion by the Sau3A restriction enzyme and two populations are formed according to the type of primer added by ligation. One is used to deplete the messengers present in the other, and vice versa. These cDNA are then used as a matrix with complementary oligonucleotides of one or the other population and amplified under conditions that preserve the representativity of each sample. After amplifications, the amplicons are mixed in a 1:4 ratio, for one and the other population. One of the two primers contains a biotinylation element in 5′. After mixing, the DNA are precipitated in ethanol, centrifuged and placed in a small reaction volume to promote renaturation, which takes place for 20 hours after a short denaturation step. Beads bound to streptavidin are added to remove the biotinylated “driver” DNA and the single-stranded fragments of the tester isolated by the addition of the SSB protein (Single Strand DNA binding protein). DNA is again added and the single-stranded DNA enrichment cycle is repeated again twice. On the DNA obtained after these steps, a PCR reaction is produced with specific oligonucleotides. This reaction enables the amplification of the targets expressed differentially. The fragments obtained are subjected to digestion by BamHI and sub-cloned in the pUC18 plasmid. After transformation and amplification, colonies are collected individually, and the plasmid DNA is extracted and subjected with the universal primer M13rev to a sequence reaction with the BigDye Teminator Cycle Sequencing kit on an ABI Prism 3730 Sequencer.

This entire procedure of cloning and identifying inserts by comparison with the data from banks such as the TIGR data (http://www;tigr.org/tigr/T_index.cgi?species=g_{—}gallus) was performed by the Genome Express company (http://www.genome-express.com/, Meylan).

### Library Screening and Cloning of the 1P06 Gene

The total RNA of the proliferating undifferentiated CESC cells are used to build a cDNA library in the open λZAP-II vector (Stratagene) in EcoRI and dephosphorylated by a CIAP treatment (Stratagene) previously described in the application FR00/06009 and Acloque et al., 2001. 150 ng of phage DNA is screened by genomic PCR using 40 μM of different oligonucleotide pairs combining either the oligonucleotide T7, or T3 present in the lZap vector and the pou(1P06)S or pou(1P06)AS oligonucleotides derived from the clone 1P06G01. The amplification conditions are denaturation for 5 minutes at 96° C., then 10 cycles of 30 seconds at 95° C., 30 seconds at 54° C. and 1 minute at 72° C. followed by 20 cycles of 30 seconds at 96° C., 30 seconds at 54° C. and 1 minute at 72° C. The last step of elongation is maintained for 7 minutes. The amplification product is subjected to agarose gel electrophoresis, purified by the Gel Extraction Kit (Qiagen), sub-cloned in the pGEM-Teasy vector (Promega) and sequenced. The pL9-4 clone contains a stop codon with 40 bp after the end of the initial 1P06g01 clone, and the pL7-2 clone enables 350 bp to be obtained upstream of the sequence.

A 5′RACE strategy is developed using the 5′RACE kit (Invitrogen). 2 mg of total RNA are reverse-transcribed for 1 hour at 42° C. with 10 pM of primer 1P06G01 (RAAS2). Once the product has been purified, a polyA tail is added by the action of the Terminal transferase TdT for 30 minutes at 37° C. (Invitrogen). ⅕ of the reaction is used as a matrix in a PCR reaction in the presence of 20 μM of the primers 1P06 (RAAS1) and AnchPrimSeq. The parameters of the reaction are denaturation at 94° C. for 2 minutes followed by 30 cycles consisting of a denaturation for 30 seconds at 94° C., hybridation for 30 seconds at 55° C. and an elongation phase of 2 minutes at 72° C. with the exception of the final reaction, which lasts for 7 minutes. A second amplification is performed on 5 ml of the product diluted 100 times using primers P06 (pL7-2)AS and PCRprimseq. The final product is purified after migration by electrophoresis, then sub-cloned in the pGEMTeasy vector. The resulting R1-8 clone contains 300 bp upstream of the pL7-2 clone with an ATG in phase. This results in an open reading frame of 888 bp.

### RT-PCR

2 mg of total RNA are reverse-transcribed for 1 hour at 42° C. using an oligodT primer and the Superscript II enzyme (Invitrogen). The real-time PCR is performed on the MXP 3000P PCR system (Stratagene). 2 ml of the mixture diluted 100 times are mixed in a final volume of 25 ml with 12.5 ml of the Mix Quantitect SYBRGreen buffer (Qiagen) in the presence of 10 μM of each primer (Proligo). Each sample is tested three times in a 96-well plate (ref. 410088 Stratagene). The parameters are: a step of denaturation for 15 min at 95° C., followed by 40 cycles consisting of denaturation for 30 seconds at 95° C., renaturation for 30 seconds at 55° and a step of elongation for 30 seconds at 72° C. The expression level of the genes is calculated by the DDCt method according to a detailed procedure available at the site: http://www.gene-quantification.info. The expression level of the ribosomal gene RS17 (X07257) is used as an internal reference between the samples.

The result of the various steps is the identification of a first fragment of 228 bp that contains a POU domain, then a sequence of 888 bp (SEQ ID N^{o}2) at the end of the library screening and 5′ RACE strategy. This sequence codes for a protein of 296 amino acids (SEQ ID N^{o}1). The POU domain of the protein thus identified begins with amino acid n^{o} 90, and is composed of 149 amino acids.

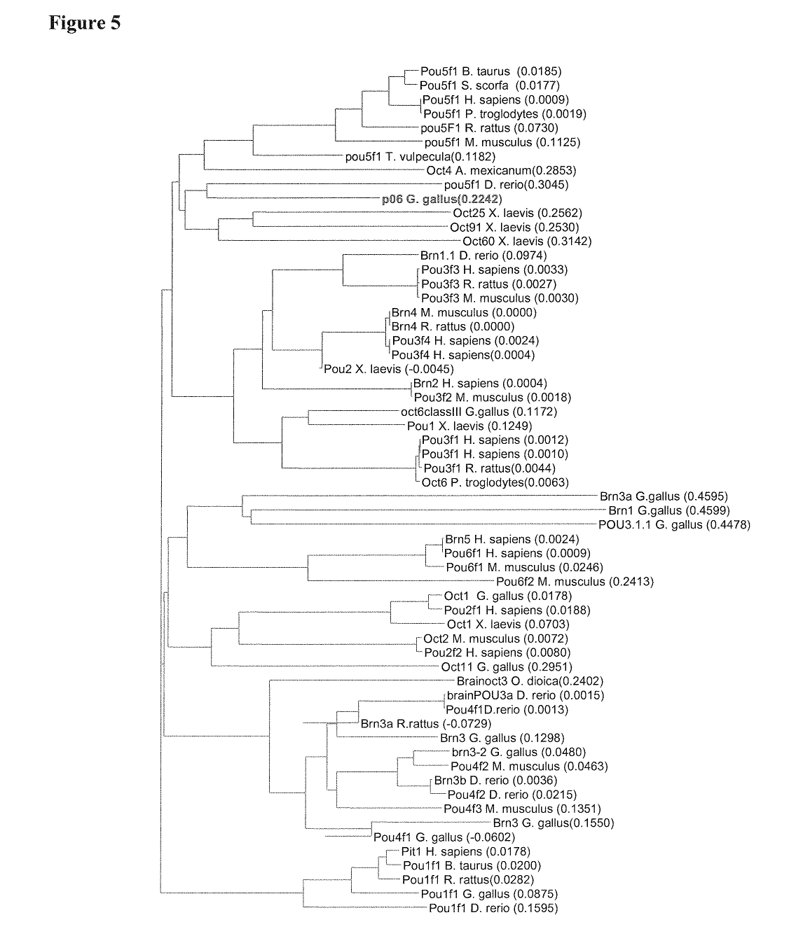

By comparing the sequences known and identified in gene data libraries of various species that include a POU domain, it is possible to represent the phylogenetic positioning of the new gene identified in the chicken, hereinafter referred to as 1P06: see FIGS. 5, 6 and 7.

Using the NTiVector software (InVitrogen), by aligning the 888 base pairs of the coding phase of the G. gallus chicken 1P06 gene with the sequences of the various Pou5f1 genes, in particular the sequences of genes with:

1155 bp tunicier O. dioica sequence (AY613856), 1134 bp of the M. musculus mouse Pou5f1 sequence (NM_{—}013633), 1615 bp of the B. taurus bovine sequence (NM_{—}174580), 1080 bp of the S. scrofa pig sequence (Q9TSV5), 1080 bp of the P. troglodytes monkey sequence (Q7YR49), 1413 bp of the H. sapiens human sequence pou5f1 (NM_{—}002701), 1141 bp of the rat R. rattus sequence (XM_{—}579282), 3111 bp of the axolot A. mexicanum sequence (AY542376.), 1418 bp zebra D. rerio fish sequence (NM_{—}131112), 570 bp of the POU domain sequence of the marsupial T. vulpecula (AY345973), 2111 bp of the Xenopus X. laevis Oct60 sequence (M60075), 2418 bp of the Xenopus X. Laevis Oct91 sequence (M60077) and 1998 bp of the Xenopus X. Laevis Oct25 sequence (M60074),

it is possible to identify that 1P06 is an ortholog of the Pou5f1 genes of the other species, because the sequence identified is closer to the sequence of these genes than the sequences of paralog genes including a POU domain (FIGS. 4, 5 and 6).

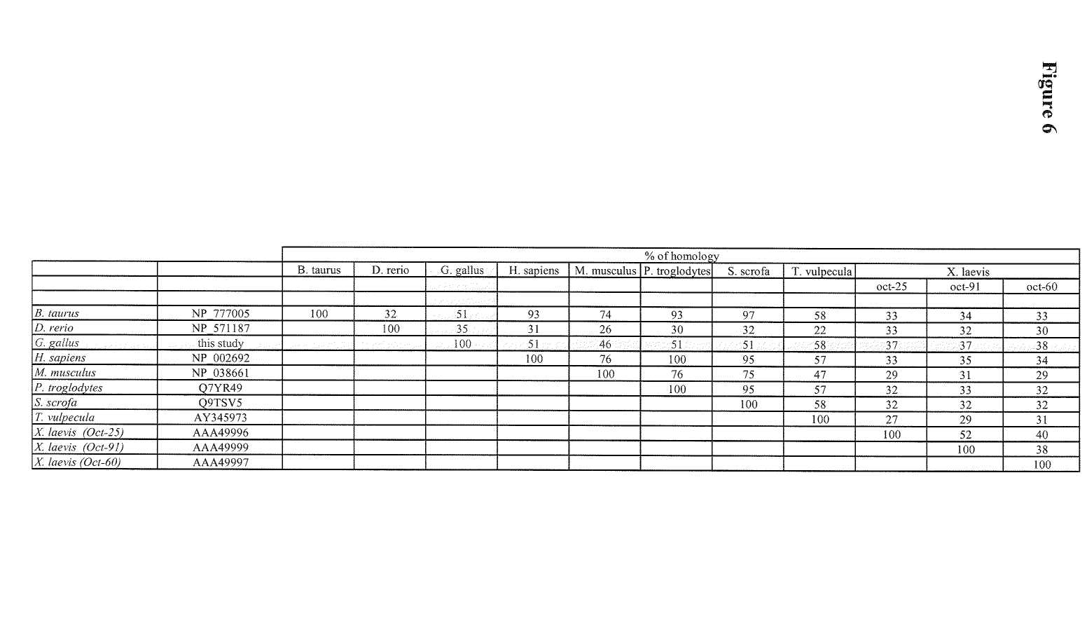

FIG. 6 shows that the chicken cDNA 1P06 has respectively 51, 46 and 51% homology with the bovine, murine and human orthologs. Percentages of homology of 35 and 38% are observed with the zebra fish gene Spg (Pou2) and various Xenopus homologs.

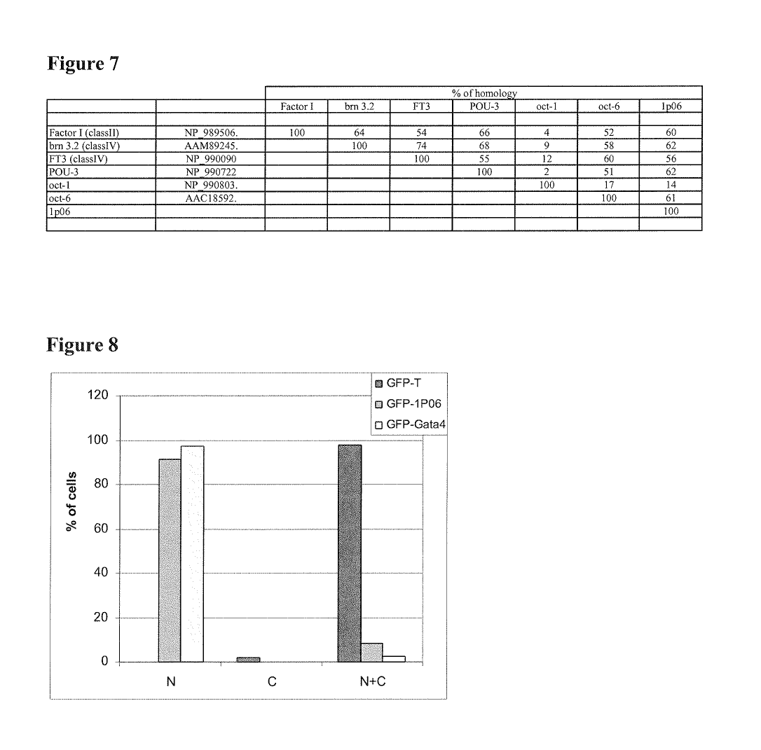

FIG. 7 shows the percentages of homology existing between the various paralogs known and identified in the chicken, and the cDNA sequence 1P06:1P06 has respectively 14, 60, 62 and 61% homology with the cDNA of Oct-1, Factor I, Brn3.2 and Oct6 (Barth et al., 1998, Levavasseur et al., 1998).

### Example 5

### Study of the Protein Encoded by the 1P06 Gene

### The GFP-1P06 Fusion Protein is a Nuclear Protein

The commercial vector pE-GFP-Cl (Clontech) was used to clone various cDNA in phase with GFP in order to see their cell localization after transfection.

We introduced the coding phases of the cDNA of 1P06, Nanog, Gcnf, Sox2, Lrh1, Sf1 and Gata4 into this vector.

The plasmids pGFP-1P06, Nanog, Gcnf, etc. were transiently introduced into the cells. The CESC cells are seeded on a sterile glass plates in wells with an average density of 15,000 to 20,000 cells per cm2. After several hours of adhesion, the transfection mixture including 1 mg of linear DNA, 3 mg of Fugene (Roche) in 100 μl of serum-free medium is prepared, left for 10 minutes at room temperature before being deposited on the cells, which are placed in the incubator for one night under normal proliferation conditions. On day 2, the transfection mixture is removed, the cells are washed with PBS then cultivated under normal conditions. After 48 hours, the cells are washed then bound with a mixture of 0.5% glutaraldehyde/1.5% formaldehyde in PBS for 15 minutes at 4° C. After washing, the cells are incubated with a Hoescht solution (10 mM/ml) so as to see the cores protected from light. The plates are mounted in the presence of a substance enabling UV rays to pass (Gel Mont BioMedia), and the fluorescence emitted by the fusion protein is observed with a fluorescence micrsocope.

The number of cells counted is n= 49 (GFP-T for control), n=71 (GFP-1P06) and n=74 (GFP-Gata4). FIG. 8 shows the percentage of cells where the observed localization of the GFP fluorescence is in the core (N), in the cytoplasm (C), or in the core and the cytoplasm (N+C). This Figure shows that the fusion protein 1P06-GFP is primarily nuclear.

Similar results have been obtained in other avian cell types such as chicken embryo fibroblasts (results not shown).

Moreover, analyses using the “band-shift” technique have made it possible to show that the 1P06 protein was capable of recognizing and binding to the DNA consensus sequence Oct-4.

To conclude, the chicken protein 1P06 is a protein with nuclear localization, which has a DNA binding activity.

### Example 6

### Overexpression of the 1P06 Gene

To evaluate the impact of the overexpression of the 1P06 gene in the physiology of CESC cells, the transfected cells with the constructions pGFP-1P06 and other fusion proteins such as GFP—SOX2, GFP-NANOG, GFP— GATA4, were analyzed from a morphological perspective.

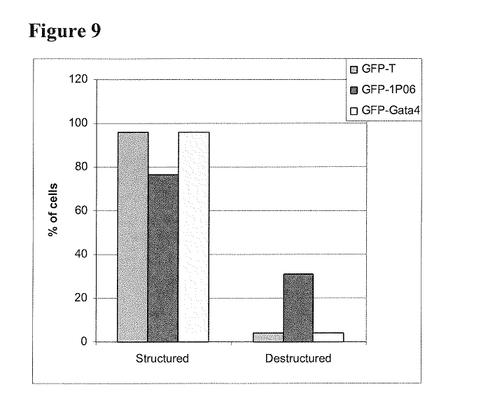

FIG. 9 shows the results obtained. The total number of cells counted is n= 51 (GFP-T control), n=93 (GFP-1P06) and n=77 (GFP-Gata4).

Most of the transfected cells do not appear to have a major modification in their morphology. An exception is observed with the construction pGFP-1P06, for which a high proportion of altered nuclear form is observed, in particular with condensed forms of the core, in around 20% of cases. The percentage of cells having a destructured chromatin is much higher in these transfected cells with the construction GFP-1P06. This may indicate apoptosis. Since the transfection time is relatively short (<72 h), it is possible to imagine being incapable of identifying major morphological and phenotypic variations under these conditions.

Our hypothesis is that the overexpression of the construction GFP-1P06 is toxic for CESC cells. A construction GFP-1P06-ERT2 was produced so as to render the nuclear localization of the fusion protein conditional. Even if the nuclear localization of the control construction GFP-ERT2 appears to be dependent on the presence of 4-hydroxytamoxifen in the culture medium, as it is expected, it was not possible to detect a major change in the localization of the fusion protein GFP-1P06-ERT2 in the presence or in the absence of 4-hydroxytamoxifen. The presence of GFP remains predominantly nuclear, regardless of the conditions. It should be assumed that the nuclear localization signal of the product 1P06 is too strong to be balanced by the presence only of the chaperone molecule that binds to the ERT2 portion in the absence of 4-hydroxytamoxifen. The same observation was made with the mutant 1P06mut, of which the mutation affects the putative localization signal nls, for which it has not been possible to obtain a major cytoplasmic signaling (Pain et al., 2004).

### Example 7

### Cloning of the Promoter of the Nanog Gene

The cloning of the promoter of the Nanog gene makes it possible to demonstrate the presence of specific transcription factor binding sites capable of regulating the expression of these genes in avian embryonic and germ stem cells.

In transfection experiments, the following results were obtained with the constructions of the Nanog promoter: this promoter is active only in proliferative cells, not differentiated cells. Thus, transient transfections in the CESC cells, undifferentiated and induced by differentiation make it possible to show that only the undifferentiated cells are capable of transactivating this promoter. Thus, transfected cells with the construction p2000Nanog-GFP show a GFP expression in 42% of the cells and only in 7% of the cells when they are treated for 48 hours by 10⁻⁷M of retinoic acid.

1. A method for preparing differentiated avian cells from avian stem cells in culture, wherein said method comprises:
inducing the differentiation of said avian stem cells by inhibiting the expression or activity of a gene expressed in said avian stem cells, wherein said gene is selected from the group consisting of 1P06, Nanog, and Eomes.

2. The method according to claim 1, wherein said inhibiting the expression or activity of the gene is performed conditionally.

3. The method according to claim 1, wherein said inhibiting the expression or activity of the gene employs at least one interfering RNA.

4. The method according to claim 3, wherein in said at least one interfering RNA comprises one or more RNAs selected from the group consisting of an antisense RNA, a double-stranded RNA, a small interfering RNA, a small hairpin RNA, and a ribosomal RNA.

5. The method according to claim 3, wherein the interfering RNA specifically inhibits the expression of the 1P06 gene.

6. The method according to claim 5, wherein the interfering RNA is encoded by a nucleic acid comprising the sequence of SEQ ID NO:3.

7. The method according to claim 3, wherein said interfering RNA specifically inhibits the expression of the Nanog gene.

8. The method according to claim 7, wherein the interfering RNA is encoded by a nucleic acid comprising the sequence of SEQ ID NO:4.

9. The method according to claim 7, wherein in the interfering RNA is encoded by a nucleic acid comprising the sequence of SEQ ID NO:5.

10. The method according to claim 3, wherein said one or more interfering RNAs specifically inhibit the expression of the Eomes gene.

11. The method according to claim 10, wherein the interfering RNA is encoded by a nucleic acid comprising a sequence selected from the group consisting of SEQ ID NO:6, NO:7, NO:8, and NO:9.

12. The method according to claim 3, wherein the interfering RNA is encoded by a nucleic acid integrated in an expression vector under the control of a promoter that enables the expression of said interfering RNA in an avian stem cell.

13. The method according to claim 3, wherein the expression of the interfering RNA is under the control of an inducible promoter.

14. The method according to claim 12, wherein said expression vector is adapted for integration into the genomes of said avian stem cells.

15. The method according to claim 1, wherein the stem cells are subjected to at least one step of activation or inhibition of specific genes to obtain a specific differentiation of said cells.

16. The method according to claim 1, wherein said avian stem cells are cultured under conditions suitable for obtaining a specific differentiation of said cells.

17. The method according to claim 1, further comprising:
selecting and destroying cells in which differentiation is not induced.

18. A composition comprising:
differentiated avian cells obtained by the method according to claim 1.

19. A method for preparing an antiviral vaccine comprising:
contacting a virus with avian stem cells that have been subjected to the method according to claim 1.

20. A composition comprising:
a nucleic acid molecule coding for an interfering RNA according to the method of claim 3.

21. The composition of claim 20, wherein
said nucleic acid molecule, is integrated in an expression vector under the control of a promoter that enables the expression of said interfering RNA in a host cell.

22. The composition of claim 21, wherein said promoter is an inducible promoter.

23. A composition comprising:
avian stem cells transformed with an expression vector according to the composition of claim 21.

24. A composition comprising:
a polypeptide comprising an amino acid sequence having at least 68% amino acid sequence identity to SEQ ID NO:1.

25. A composition comprising:
a nucleic acid encoding a polypeptide according to the composition of claim 24.

26. A composition comprising:
a nucleic acid that comprises a sequence having at least 94% nucleic acid sequence identity to SEQ ID NO:2.

27. A composition comprising:
an expression vector comprising a nucleic acid molecule according to the composition of claim 25.

28. A composition comprising:
a nucleic acid that comprises a sequence having at least 90% nucleic acid sequence identity to SEQ ID NO:11.

29. A composition comprising:
an expression vector comprising a nucleic acid molecule according to the composition of claim 28.

30. A composition comprising:
a prokaryotic or eukaryotic cell having an expression vector according to the composition of claim 27.

31. A composition comprising:
an expression vector comprising a nucleic acid molecule according to the composition of claim 26.

32. A composition comprising:
a prokaryotic or eukaryotic cell having an expression vector according to the composition of claim 29.
